(19) United States
(12) Patent Application Publication
(10) Pub. No.: US 2023/0331604 A1
(43) Pub. Date: Oct. 19, 2023

(54) METHOD FOR THE ELECTROCHEMICAL CONTROL OF THE PHOTOSYNTHETIC METABOLISM OF PURPLE NON-SULFUR BACTERIA AND REDOX MEDIATORS THEREOF

(71) Applicant: Zuccato HC S.r.l., Verona (IT)

(72) Inventors: Paolo FACCI, Bogliasco (GE) (IT); Alessandro ZUCCATO, Sona (VR) (IT)

(21) Appl. No.: 18/027,158

(22) PCT Filed: Sep. 22, 2021
(86) PCT No.: PCT/IB2021/058634

§ 371 Date: Mar. 20, 2023

(30) Foreign Application Priority Data
Sep. 24, 2020 (IT) 102020000022594

(51) Int. Cl. C02F 3/00

(52) U.S. Cl. CPC C02F 3/005; C02F 2103/32

(57) ABSTRACT

The present invention relates to a method for the electrochemical control of the level of gene expression in purple non-sulfur bacteria, in particular Rhodobacter, and relative applications for the treatment of wastewater. The invention also relates to the use of fat-soluble redox mediators capable of permeabilizing the bacterial membrane and altering the oxidation state of the disulfide bond present in thioredoxins.

The present invention relates to a method for the electrochemical control of the level of gene expression in purple non-sulfur bacteria, in particular Rhodobacter, and relative applications for the treatment of wastewater. The invention also relates to the use of fat-soluble redox mediators capable of permeabilizing the bacterial membrane and altering the oxidation state of the disulfide bond present in thioredoxins.

Purple non-sulfur bacteria (Athiorodaceae) are the only photosynthetic bacteria that are not strictly anaerobic. The most representative genera of purple non-sulfur bacteria are Rhodobacter, Rhodopseudomonas and Rhodospirillum.

Some species tolerate oxygen and can grow in the dark, under aerobic conditions, drawing energy from the respiratory metabolism of organic compounds; consequently, unlike other photosynthetic bacteria, the organisms belonging to these species are not necessarily photosynthetic, but optional.

The synthesis of photosynthetic pigments in this group of purple bacteria is specifically inhibited by oxygen in relatively low concentrations, regardless of the presence or absence of light; consequently, the introduction of air into a growing culture leads to a progressive decrease in the cellular content of bacteriochlorophyll and carotenoids, so that the cells end up becoming almost completely colourless.

The various species in the group can use a wide range of substrates, which includes many fatty acids, primary and secondary alcohols, dicarboxylic acids and other organic acids, carbohydrates and aromatic compounds.

With rare exceptions, any organic compound capable of supporting the photosynthetic growth of purple sulfur-free bacteria under anaerobic conditions will be used for growth by the same organism even under aerobic conditions.

The metabolic sequences of photosynthesis are, however, completely different from those of respiration.

In respiration, a large part of the carbon of the organic substrate is completely oxidized to CO₂ through the cycle of tricarboxylic acids, with the generation of ATP through oxidative phosphorylation reactions.

Under anaerobic conditions and in the presence of light, the cyclic photophosphorylation reactions generate a potentially unlimited amount of ATP to be used in biosynthesis, thus allowing an almost total assimilation of the carbon contained in the organic substrates.

Under photosynthetic conditions, little or no amount of organic substrate is oxidized through the cycle of tricarboxylic acids; in the anaerobic photometabolism of most organic substrates, this cycle is of secondary importance.

During the anaerobic metabolism of organic substrates, in the presence of light, the redox balance is maintained either by oxidation of part of the substrate to CO₂, if the substrate is more oxidized than the cellular material, or by a concomitant assimilation and reduction of CO₂, if the organic substrate is smaller than the cellular material.

Roughly speaking, the photosynthetic metabolism of organic compounds carried out by purple bacteria can be schematized as follows:

light+organic substrate+CO₂→organic cellular material

The biochemical mechanisms involved can be illustrated by considering the photometabolism of two fatty acids: acetate, slightly more oxidized than cellular material, and butyrate, slightly more reduced.

The assimilation of both of these fatty acids leads, first of all, to the formation of a cellular reserve substance, poly-beta-hydroxybutyrate, through the sequences of reactions illustrated hereunder.

More specifically, the general transformation of acetate into poly-beta-hydroxybutyrate is a reductive process:

2nCH₃COOH+2nH→(C₄H₆O₂)ₙ+2nH₂O

The necessary reducing power is produced anaerobically by the concomitant oxidation of part of the acetate, through the reactions of the tricarboxylic acid cycle, according to the general equation:

CH₃COOH+2H₂O→2CO₂+8H

The overall equation for these two acetate metabolism pathways is:

9nCH₃COOH→4(C₄H₆O₂)ₙ+2nCO₂+2nH₂O

The assimilation is therefore extremely efficient; almost 90% of the carbon of the acetate is in fact assimilated thanks to the generation of ATP, necessary for the assimilative process, in cyclic photophosphorylation.

The carbon assimilation of the acetate becomes almost total if molecular hydrogen is supplied as an external reducing power source.

The transformation of butyrate into poly-beta-hydroxybutyrate on the other hand, corresponds to an oxidation:

nCH₃CH₂CH₂COOH→(C₄H₆O₂)ₙ+2nH

During the photosynthetic assimilation of this fatty acid, the excess reducing power produced is used for a coupled assimilation of CO₂ through Calvin cycle reactions, which gives a polysaccharide as its main product, whose general formula is (CH₂O):

nCO₂+4nH→(CH₂O)ₙ+nH₂O

During the photometabolism of butyrate, a complete assimilation of the carbon of the organic substrate takes place, always coupled with the assimilation of CO₂:

nC₄H₈O₂+nCO₂→(C₄H₆O₂)ₙ+(CH₂O)ₙ

Thanks to this metabolic versatility, the purple optional photosynthetic bacteria are fundamental in the biodegradation process of organic compounds in the soil and in aquatic environments.

As already mentioned, in purple optional photosynthetic bacteria, the metabolism can be sustained by oxidative phosphorylation reactions if in the presence of oxygen, or by cyclic (photosynthetic) phosphorylation reactions if the oxygen tension is insufficient and if there is light radiation as free energy source.

The reasons for the alternative possibilities (respiration or photosynthesis) that these bacteria have for synthesizing what is needed for their survival and duplication lie in their genetics.

It has in fact been demonstrated that the oxygen tension acts as a trigger parameter capable of modulating the expression level of the genes responsible for the production of photosynthesis proteins [1].

In particular, in-depth studies [1] on bacteria of the Rhodobacter type (capsulatus, sphaeroides) have identified in the specific chain of biomolecular reactions involved in the expression of these genes, the fundamental role of a family of enzymes, thioredoxins, in modulating the response to changes in the oxygen tension by these bacteria.

Thioredoxins (type A and C) are enzymes involved in the catalysis of binding reactions between other bacterial enzymes such as gyrase, and the double-stranded DNA of the bacterial chromosome. Thioredoxins change their structure and consequently their functional capacities as a result of a variation in the oxidative state of the environment in which they are found (bacterial cytoplasm). Their structure is in fact characterized by the presence of a functional S—S bond exposed to the solvent, which oxidizes or reduces according to the oxidative state of the environment to which it is exposed. This difference in the structure generates fundamental consequences in the capacity of thioredoxins of binding to the gyrase and therefore assisting its topoisomerase activity, which is expressed in a variation in the degree of negative superelicity of the bacterial DNA. This, in turn, modulates the accessibility of the DNA by RNA-polymerases and therefore the probability of transcription of the genes corresponding to the stretch of DNA in question.

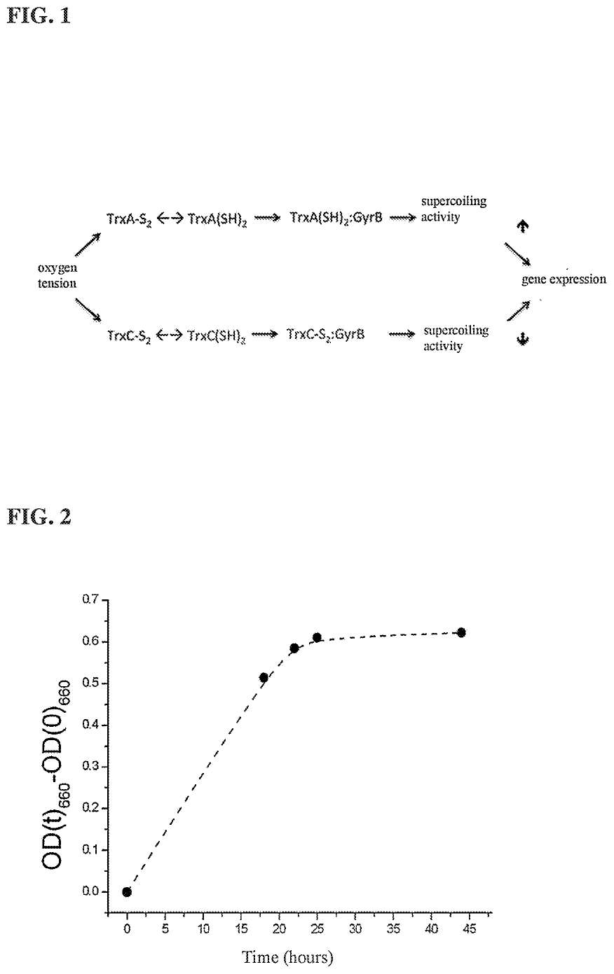

The diagram of FIG. 1 summarizes this typical behaviour of Rhodobacter (it should be noted that in R. sphaeroides only thioredoxin type A is present, whereas in R. capsulatus, thioredoxin type A and C is present) [1].

It can be seen how the oxygen tension acts differently on the thioredoxin type A and C, with the result of modulating the activity of gyrases in the same direction, and therefore the accessibility to certain sections of the bacterial chromosome by RNA-polymerases. The photosynthetic metabolism is naturally necessarily supported by the presence of sufficient light radiation.

The concurrence of the two conditions set out so far, namely:
i) the particular efficiency in using the oxidizable carbonaceous substrate present in the environment in the case of photosynthesis by purple optional photosynthetic bacteria and
ii) the possibility of modulating the expression level of photosynthetic genes through the oxidative state of the bacterial cytoplasm,
   has led the authors of the present invention to develop a method for the electrochemical control of the level of gene expression in these optional photosynthetic bacteria, regardless of the presence of atmospheric oxygen, in order to force the metabolism of purple non-sulfur bacteria (Rhodobacter) towards photosynthesis, thus optimizing the use of the carbonaceous substrate present in the environment.

This is enabled by the use of suitable fat-soluble redox mediators ([2], [3]) capable of permeabilizing the bacterial membrane and entering the cytoplasm, in order to alter the oxidation state of the disulfide bond present in type A thioredoxins, as well as by appropriate light irradiation. In order to do this, it is essential to use molecules that have a more negative equilibrium potential than the corresponding thioredoxin potential, the latter being typically around −460 mV (vs Saturated Calomel Electrode or SCE reference electrode).

If in fact the bacterial cytoplasm experiences a reducing condition, in the presence of light radiation, it promotes the expression of photosynthetic genes to the detriment of those of the respiratory chain [1].

This system, which can be easily controlled by using suitable redox mediators, can be advantageously used for the treatment of wastewater of an industrial food origin for reducing the excess oxidizable carbon present therein.

The present invention therefore relates to a method for the electrochemical control of the photosynthetic metabolism in purple non-sulfur bacteria comprising the culture of a population of purple non-sulfur bacteria in an electrochemical cell in the presence of an irradiation source and a redox mediator in the solution of the fermenter having a more negative equilibrium potential than that of cytoplasmic thioredoxins (−460 mV) capable of reducing the disulfide bonds of said thioredoxins, said electrochemical cell being characterized by the presence of a working electrode, a counter electrode and, optionally, a reference electrode; wherein said working electrode has a more negative equilibrium potential than the equilibrium potential of the redox mediator.

Once reduced by the redox mediator, the cytoplasmic thioredoxins of purple non-sulfur bacteria will interact with the B gyrases and promote the expression of photosynthetic genes at the expense of respiratory genes after altering the supercoiling state of the bacterial DNA.

The method for the electrochemical control of the photosynthetic metabolism in purple non-sulfur bacteria according to the invention can be carried out regardless of the presence of atmospheric oxygen.

The electrochemical cell can preferably be included in a bioreactor or fermenter.

The purple non-sulfur bacteria can belong to the genera Rhodobacter, Rhodopseudomonas, Rhodospirillum. The bacteria preferably belong to the Rhodobacter genus and even more preferably to the Rhodobacter sphaeroides or Rhodobacter capsulatus species.

The thioredoxins can be type A or C, depending on the type of bacterium. In R. sphaeroides for example, only type A thioredoxin is present, whereas in R. capsulatus, both type A and type C thioredoxins are present).

The working electrode of the electrochemical cell can be of gold, platinum, stainless steel. The counter electrode can be of graphite or stainless steel.

The reference electrode can be selected from hydrogen electrode (SHE), SCE or Ag/AgCl at various saturation levels.

According to a preferred embodiment of the method according to the invention, the redox mediator having an equilibrium potential more negative than that of thioredoxins (about −460 mV) is selected from the group of safranins, i.e. the azines of 2,8-dimethyl-3,7-diaminophenazine having an equilibrium potential of approximately −540 mV. The redox mediator is preferably safranin T having the following formula:

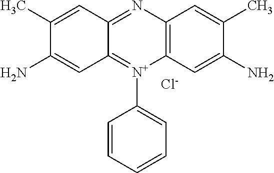 [SMILES: Cc1cc2nc3cc(C)c(N)cc3[n+](-c3ccccc3)c2cc1N ; [Cl-]]

and an equilibrium potential of −540 mV.

According to a preferred embodiment of the method according to the invention, the redox mediator is added in the electrochemical cell at a concentration ranging from 25 nM to 250 nM, preferably at a concentration equal to 50 nM.

The concentration of the redox mediator within the fermenter/reactor solution has proved to be critical. If the concentrations are too low (<10 nM), the mediator is ineffective, whereas excessively high concentrations (>500 nM) tend to kill the bacteria.

Again according to a preferred embodiment, the redox mediator molecules are kept under mild stirring in the solution inside the fermenter/reactor in order to favour recirculation.

The invention also relates to an electrochemical cell comprising a culture of purple non-sulfur bacteria, a redox mediator with an equilibrium potential lower than −460 mV, a working electrode, a counter electrode and, optionally, a reference electrode, wherein said working electrode has a more negative equilibrium potential than the equilibrium potential of the redox mediator.

The working electrode of the electrochemical cell can be of gold, platinum, stainless steel. The counter electrode can be of graphite or stainless steel.

The working electrode is preferably of stainless steel; the counter electrode is a graphite bar. The reference electrode can be selected from hydrogen electrode (SHE), SCE or Ag/AgCl at various saturation levels.

The present invention also relates to the use of safranins as redox mediators for the electrochemical control of the photosynthetic metabolism of purple non-sulfur bacteria in the presence of an irradiation source.

According to a preferred embodiment, the redox mediator is safranin T having the following formula:

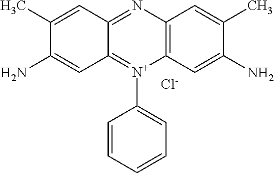 [SMILES: Cc1cc2nc3cc(C)c(N)cc3[n+](-c3ccccc3)c2cc1N ; [Cl-]]

and an equilibrium potential of −540 mV.

The concentration of use of the safranins ranges from 25 nM to 250 nM, and is preferably 50 nM.

In a further preferred embodiment, the working electrode has an equilibrium potential of about −660 mV.

The invention further relates to a method for the chemical oxidation of a substrate comprising one or more organic compounds of carbon, comprising the following steps:
(a) cultivating purple non-sulfur bacteria in an electrochemical cell of a reactor in the presence of an irradiation source and a redox mediator having an equilibrium potential more negative than cytoplasmic thioredoxins of purple non-sulfur bacteria; wherein said electrochemical cell is characterized by the presence of a working electrode, a counter electrode and, optionally, a reference electrode; wherein said working electrode has an equilibrium potential more negative than the redox mediator,
(b) putting the purple non-sulfur bacteria with the activated photosynthetic metabolism in contact with a substrate comprising one or more organic compounds of carbon.

Once reduced, the cytoplasmic thioredoxins of step a) will interact with the B gyrases and promote the expression of photosynthetic genes to the detriment of respiratory genes after having altered the supercoiling state of the bacterial DNA. The cyclic phosphorylation biochemical reactions will be more efficient than the oxidative phosphorylation reactions in using the organic carbon available.

The purple non-sulfur bacteria can belong to the Rhodobacter. Rhodopseudomonas. Rhodospirillum genera. The bacteria preferably belong to the Rhodobacter genus and even more preferably to the Rhodobacter sphaeroides or Rhodobacter capsulatus species.

According to an alternative embodiment of the chemical oxidation method of the invention, the above substrate is a liquid. The substrate is even more preferably wastewater of industrial food, tanning or civil origin.

The organic carbon compounds present in the substrate can be of various kinds and in particular can be selected from fatty acids, primary and secondary alcohols (e.g. methanol, ethanol, propanol, butanol or isobutanol), dicarboxylic acids, other organic acids (e.g. lactic acid, citric acid, gluconic acid), carbohydrates (such as sugars) and aromatic compounds (such as chlorophenols).

In a preferred embodiment the redox mediator is safranin T having an equilibrium potential of about −540 mV.

In a further preferred embodiment the working electrode has an equilibrium potential of about −660 mV.

The redox mediator is even more preferably safranin T at a concentration of 50 nM.

According to a further preferred embodiment, the working electrode is a stainless steel grid; the counter electrode is a graphite bar.

The reference electrode, if present, can be hydrogen (SHE), (SCE) or Ag/AgCl at various saturation levels, preferably SCE.

The present invention will now be described for illustrative, but non-limiting, purposes according to a preferred embodiment with particular reference to the attached figures, wherein:

FIG. 1 shows the action scheme of thioredoxins, on the part of gyrase B, in relation to the oxygen tension in Rhodobacter.

FIG. 2 illustrates the aerobic growth curve of R. sphaeroides in M550 wherein the ordinates show the absorbance at 660 nm at time t-at time 0, and in the abscissas the hours that have elapsed since inoculation.

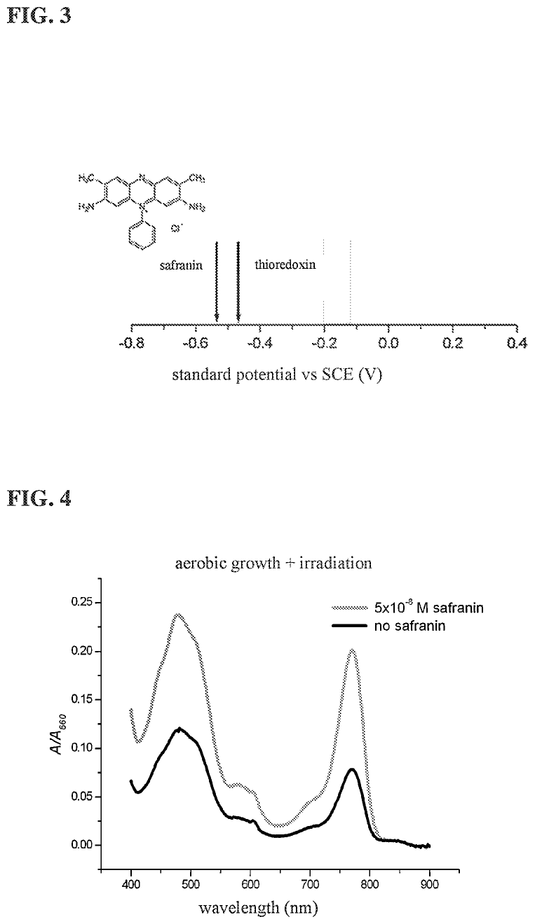

FIG. 3 illustrates the safranin formula and a diagram with the equilibrium potentials of safranin and thioredoxin.

FIG. 4 shows the optical absorption spectra of bacteriochlorophyll extracted from two different preparations of R. sphaeroides.

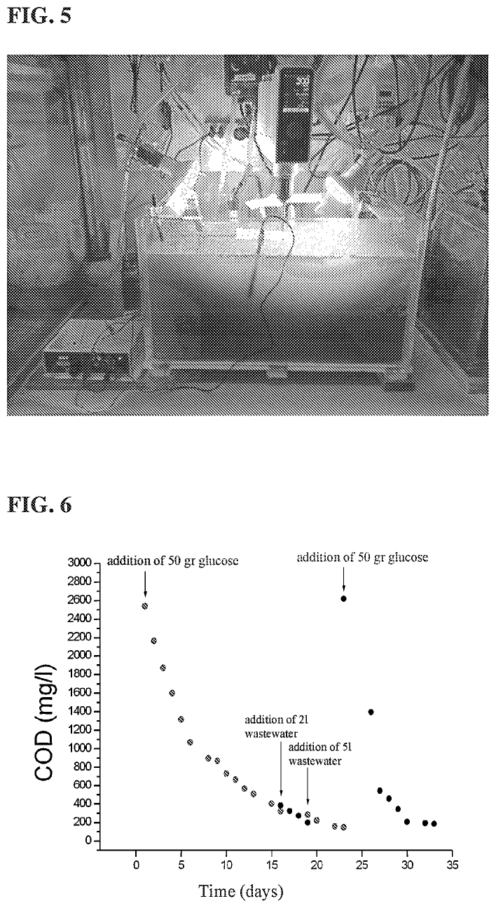

FIG. 5 exemplifies an experimental setup of the reactor used for the abatement of c.o.d. (chemical oxygen demand) in wastewater using the method according to the invention.

FIG. 6 shows the trend of the c.o.d. (chemical oxygen demand) in relation to time and for different initial dosages of c.o.d. (sugar or addition of a known volume and c.o.d. effluents).

The following non-limiting examples are now provided for a better illustration of the invention wherein the authors have implemented an electrochemical control method of the level of gene expression in Rhodobacter.

### EXAMPLE 1: PREPARATION METHOD FOR PROMOTING THE EXPRESSION OF PHOTOSYNTHETIC GENES IN RHODOBACTER TO THE DETRIMENT OF THOSE OF THE RESPIRATORY CHAIN

A strain of Rhodobacter sphaeroides (ATCC® 55304 ™) was used whose growth curve in optimized medium M550 is shown in FIG. 2.

A homemade potentiostat was used as an external source of free energy, capable of providing a current of up to 100 mA and keeping the potential constant within the range of −2 V÷+2 V).

Safranin T was used for reducing the disulfide bonds of the thioredoxins (which has an equilibrium potential of about −540 mV (vs SCE, reference electrode), i.e. more negative than the corresponding potential of the thioredoxins which typically is around −460 mV (vs SCE), as shown in FIG. 3.

Using these molecules inside a reactor in which a working electrode, a counter electrode and a possible reference electrode are also inserted and keeping the working electrode at a more negative potential than the equilibrium potential of the mediator, the latter will remain reduced and will thus be capable of reducing the disulfide bonds of the cytoplasmic thioredoxins, regardless of the presence or absence of atmospheric oxygen in the fermenter.

Thanks to the diffusion, the redox mediator, after conditioning the oxidative state of the bacterial cytoplasm, will be able to come back into contact with the working electrode and will therefore be reduced again by the working electrode kept at a negative potential by the potentiostat, being recharged with electrons and being able to repeat the cycle described.

The materials of the electrodes used are:
Working electrode: stainless steel grid with 1 mm mesh spacing.
Counter electrode: graphite bars (0.8 cm in diameter, 30 cm long)
Reference electrode: (SCE)

Safranin T, molecular mass equal to 350.85. (Fluka) at a concentration equal to 50 nM, was used as redox mediator in the cell.

The solution inside the fermenter/reactor was kept under mild stirring to facilitate the recirculation of the redox mediator molecules.

The overall ionic strength of the solution must be >10 mM to allow the potentiostat to control the potential of the working electrode [4].

The periodic dosage of vitamins such as biotin (0.06-0.6 mg/1), nicotinamide (1-10 mg/1), nicotinic acid (1-10 mg/1) and thiamine (2-20 mg/i) is also essential for supporting cell duplication during the increase in the biomass and the functioning of the system.

A comparison between the quantity of bacteriochlorophyll, an essential component of photosynthetic pigments (reaction centers, antenna pigments LHI and LHII) synthesized by the population of Rhodobacter sphaeroides subjected to the electrochemical control conditions with the above parameters, and that produced by a population of the same bacteria but maintained in aerobic growth, is indicated in the data shown in the graph of FIG. 4. From the absorption spectra it can be observed that the absorbance of the solution obtained from the processing of the bacterial suspension with acetone/methanol, normalized to that at 660 nm (proportional to the bacterial number for R. sphaeroides) shows the absorption bands of bacteriochlorophyll. The intensity of the bands of the population subjected to electrochemical control in the presence of 50 nM safranin appears almost tripled compared to the corresponding aerobic population.

This result indicates that the use of these conditions actually simulates a shortage of oxygen that induces the overexpression of photosynthetic genes. In other words, these results indicate the capacity of the technology proposed of electrochemically modulating the expression level of Rhodobacter genes (photosynthesis vs respiration).

### EXAMPLE 2: USE OF THE ELECTROCHEMICAL MODULATION TECHNOLOGY OF THE EXPRESSION LEVEL OF RHODOBACTER GENES FOR WASTEWATER TREATMENT

The technology illustrated in Example 1 was used for reducing the c.o.d. (chemical oxygen demand) value in solutions of a known composition or following the addition of the volume of wastewater of an industrial food origin as an example of the effectiveness of the method found.

After growing the biomass of R. sphaeroides (ATCC® 55304™) up to desired values within the range of values of sst-total suspended solids—equal to 1-5 g/l, degradation kinetic measurements of the c.o.d. were carried out in relation to time. After reaching a threshold value of 200-300 mg/l of c.o.d., the reactor was enriched again by dosing sugar or adding known volumes of wastewater with a known c.o.d value.

The reactor used (FIG. 5) was configured with the following parameters:
Reactor volume: 40 l
T=28° C.
pH=7
Redox potential reactor solution=−300÷−400 mV
SST=1÷0.3 g/l
SSV=0.9÷0.28 g/l
NH₄⁺=8÷10 mg/l
NO₂=0
NO₃=0
PO₄⁺>100 mg/l
V_{WE}=−660 mV (vs SCE)
Safranin concentration=50 nM
Working electrode: stainless steel grid
Counter electrode: graphite bar
Reference electrode: SCE

The reactor is continuously irradiated during the whole operating period by 3 LED lamps (15 W, T=6400K, Aigostar) kept at a distance of 30 cm from the upper surface.

FIG. 6 shows a graph of the trend of the c.o.d. in relation to time for a reactor configured as described above. It can be noted that the abatement of c.o.d. follows a “sawtooth” trend with discontinuity in correspondence with the various additions of oxidizable carbon (sugar or wastewater). The abatement kinetics are naturally faster in correspondence with high values of c.o.d. (corresponding to abundant nourishment available for the bacteria), but it can be observed how the c.o.d. abatement rate increases with the same c.o.d. values in the various subsequent cycles, indicating an adaptation of the bacterial population to environmental conditions. Whereas, in fact, in the initial (linear) part of the first cycle the abatement rate was 294 mg/l per day, in the cycle relating to the second dosage of c.o.d. this rate becomes equal to 482 mg/l per day with a substantially equal biomass present (approximately 1 g/l).

These preliminary results suggest that R. sphaeroides, when subjected to electrochemical control in the presence of light irradiation and an appropriate concentration of fat-soluble redox mediator and with a more negative equilibrium potential than that of cytoplasmic thioredoxins, behaves as an excellent c.o.d. reducer.

Furthermore, under the conditions used, no excess biomass is produced and no forced aeration is required by the microorganism, unlike the activated sludge composed of cocktails of aerobic microorganisms commonly used in the purifiers of the state of the art.

### BIBLIOGRAPHY

[1] Kuanyu Li et al., Nucleic Acids Research, 2004, 32, 4563-4575.
[2] E. Katz, A. N. Shipway and I. Willner Biochemical fuel cells in Handbook of Fuel Cells—Fundamentals, Technology and Applications, Eds. Wolf Vielstich.
[3] Hubert A. Gasteiger. Arnold Lamm. Vol. 1: Fundamentals and Survey of Systems. 2003 John Wiley & Sons, Ltd.
[4] A. Bard & L. Faulkner Electrochemical Applications, second Edition, Wiley, 2001.

1. A method for the electrochemical control of the photosynthetic metabolism of purple non-sulfur bacteria using an electrochemical cell, said electrochemical cell comprising a working electrode, a counter electrode and, optionally, a reference electrode in the presence of an irradiation source, said method comprising adding a redox mediator having an equilibrium potential more negative than cytoplasmic thioredoxins in said electrochemical cell, wherein said working electrode has an equilibrium potential more negative than the redox mediator.

2. The method for the electrochemical control of the photosynthetic metabolism of purple non-sulfur bacteria according to claim 1, wherein said purple non-sulfur bacteria are selected from the group consisting of Rhodobacter, Rhodopseudomonas and Rhodospirillum.

3. The method for the electrochemical control of the photosynthetic metabolism of purple non-sulfur bacteria according to claim 2, wherein said purple non-sulfur bacteria belong to the Rhodobacter sphaeroides or Rhodobacter capsulatus species.

4. The method for the electrochemical control of the photosynthetic metabolism of purple non-sulfur bacteria according to claim 1, wherein the working electrode of the electrochemical cell is selected from the group consisting of gold, platinum and stainless steel; the counter electrode is selected from graphite and stainless steel, and the reference electrode, if present, is selected from the group consisting of hydrogen electrode (SHE), SCE and Ag/AgCl.

5. The method for the electrochemical control of the photosynthetic metabolism of purple non-sulfur bacteria according to claim 1, wherein the redox mediator is a safranin.

6. The method for the electrochemical control of the photosynthetic metabolism of purple non-sulfur bacteria according to claim 1, wherein the redox mediator is added in the electrochemical cell at a concentration ranging from 25 nM to 250 nM.

7. The method for the electrochemical control of the photosynthetic metabolism of purple non-sulfur bacteria according to claim 1, wherein the working electrode has an equilibrium potential of −660 mV.

8. An electrochemical cell comprising a culture of purple non-sulfur bacteria, a redox mediator with an equilibrium potential of less than −460 mV, a working electrode, a counter electrode and, optionally, a reference electrode; wherein said working electrode has an equilibrium potential more negative than the redox mediator.

9. The electrochemical cell according to claim 8, wherein the redox mediator is a safranin.

10. The electrochemical cell according to claim 8,
wherein the working electrode is selected from the group consisting of gold, platinum and stainless steel; the counter electrode is selected from the group consisting of graphite and stainless steel, and the reference electrode, if present, is selected from the group consisting of hydrogen electrode (SHE), SCE and Ag/AgCl.

11-12. (canceled)

13. A method for the chemical oxidation of a substrate comprising one or more carbon compounds, comprising:
(a) cultivating purple non-sulfur bacteria in an electrochemical cell of a reactor in the presence of an irradiation source and a redox mediator having an equilibrium potential more negative than the cytoplasmic thioredoxins of purple non-sulfur bacteria; wherein said electrochemical cell is characterized by the presence of a working electrode, a counter electrode and, optionally, a reference electrode; wherein said working electrode has an equilibrium potential more negative than the redox mediator; and
(b) putting purple non-sulfur bacteria with an activated photosynthetic metabolism in contact with a substrate comprising one or more carbon compounds.

14. The method for the chemical oxidation of a substrate according to claim 13, wherein said purple non-sulfur bacteria is selected from the group consisting of Rhodobacter, Rhodopseudomonas and Rhodospirillum, preferably the Rhodobacter sphaeroides and Rhodobacter capsulatus species.

15. The method for the chemical oxidation of a substrate according to claim 13, wherein said substrate is a liquid.

16. The method for the chemical oxidation of a substrate according to claim 13, wherein the redox mediator is a safranin.

17. The method for the chemical oxidation of a substrate according to claim 13, wherein the redox mediator is added at a concentration ranging from 25 nM to 250 nM.

18. The method for the chemical oxidation of a substrate according to claim 5, wherein the safranin is safranin T having the following formula: 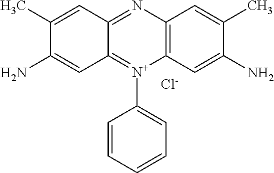 [SMILES: Cc1cc2nc3cc(C)c(N)cc3[n+](-c3ccccc3)c2cc1N ; [Cl-]]
and an equilibrium potential of −540 mV.

19. The method for the electrochemical control of the photosynthetic metabolism of purple non-sulfur bacteria according to claim 6, wherein the redox mediator is added in the electrochemical cell at a concentration of 50 nM.

20. The electrochemical cell according to claim 9, wherein the safranin is safranin T having the following formula: 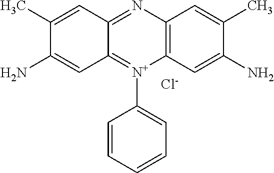 [SMILES: Cc1cc2nc3cc(C)c(N)cc3[n+](-c3ccccc3)c2cc1N ; [Cl-]]
and an equilibrium potential of −540 mV.

21. The method for the chemical oxidation of a substrate according to claim 15, wherein the liquid is a wastewater of the food industry.

22. The method for the chemical oxidation of a substrate according to claim 16, wherein the safranin is safranin T having the following formula: 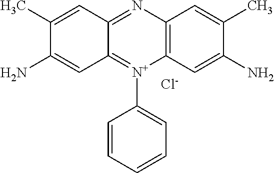 [SMILES: Cc1cc2nc3cc(C)c(N)cc3[n+](-c3ccccc3)c2cc1N ; [Cl-]]
and an equilibrium potential of −540 mV.

23. The method for the chemical oxidation of a substrate according to claim 17, wherein the redox mediator is added at a concentration of 50 nM.
